(19) United States
(12) Patent Application Publication
(10) Pub. No.: US 2019/0142614 A1
(43) Pub. Date: May 16, 2019

(54) System and adapter device for fixing a cover to a prosthetic limb

(71) Applicant: Anatomic Studios Sweden AB, Malmo (SE)

(72) Inventors: Staffan DAHLBERG, Malmo (SE); Stephanie FRANKSSON, Orby (SE); Axel NORDIN, Malmo (SE); Malkus ARLEMARK, Malmo (SE)

(21) Appl. No.: 16/308,744

(22) PCT Filed: Jun. 16, 2017
(86) PCT No.: PCT/SE2017/050651

§ 371 Date: Dec. 10, 2018

(30) Foreign Application Priority Data
Jun. 27, 2016 (SE) 1650918-4

(51) Int. Cl. A61F 2/76; A61F 2/50; A61F 2/68; A61F 2/60

(52) U.S. Cl. CPC A61F 2/76; A61F 2/5046; A61F 2002/707; A61F 2/60; A61F 2/68

(57) ABSTRACT

A prosthesis system (10) adapted for being worn by a user (1) comprises at least a first cover (11), a prosthetic limb (12) and an adapter device (13, 14) for fixing the first cover to the prosthetic limb, wherein the adapter device comprises a first adapter part (13) configured for being attached to the first cover, a second adapter part (14) configured for being mounted onto the prosthetic limb and locking means (131, 141) for locking the cover (11) onto the prosthetic limb.

### TECHNICAL FIELD

The present disclosure relates to the field of prosthetics for limbs such as legs and arms. More particular the invention pertains to a prosthetic system adapted for fixing a cover to a prosthetic limb by using an adapter device.

### BACKGROUND ART

Today, around 6000 leg amputees live in Sweden and about 8,000 leg amputations are performed per year. Most get a prosthesis which they are happy with from a purely functional perspective, but the psychological aspect that comes when one loses a part of oneself is often ignored in the design of the prosthesis. A growing group of amputees, especially young and active, are asking for prostheses that are not reminiscent of a hospital setting and something they want to hide, but instead something that gives them more confidence, such as a prosthesis which rather looks like a fashion accessory than an aid.

Most prosthetic limb solutions are individually designed to gain optimal fitting and function. Cosmetic solutions will therefore also involve individual design to achieve best appearances with relative high costs as a result. Consequently most users are given no or few options to decide the cosmetic appearance of their prosthesis. This is partly due to costs involved with single-unit manufacturing. Recently, additive methods have been developed which has reduced the cost of prototyping and given greater freedom in the design language, but it is still an expensive process, and there are still requirements such as strength, weight, cosmetic fit with the user's healthy limb and prosthesis movement that need to be taken into consideration. Some of these problems can be facilitated by 3D scanning of the user's healthy limb and existing prosthesis, but it still requires many hours of work by orthopaedic engineers and designers, which greatly increases the cost to create a finished cosmetic solution that is ready for manufacturing.

### SUMMARY OF THE INVENTION

An object of the present disclosure is to provide a system and device for fixing a cover to a prosthetic limb which seek to mitigate, alleviate, or eliminate one or more of the above-identified deficiencies in the art and disadvantages singly or in any combination.

The present disclosure is defined by the appended claims. Various advantageous embodiments of the disclosure are set forth by the appended claims as well as by the following description and the accompanying drawings.

The object is obtained by a prosthesis system adapted for being worn by a user comprising at least a first cover and a prosthetic limb, wherein the system further comprises:
an adapter device for fixing the first cover to the prosthetic limb comprising:
   a first adapter part configured for being attached to the first cover;
   a second adapter part configured for being mounted onto the prosthetic limb; and
   locking means for locking the first adapter part onto the second adapter part when the first and second adapter parts are connected.

In some aspect a connecting surface of the first adapter part is inclined and a corresponding connecting surface of the second adapter part is inclined in the opposite direction according to the first adapter part.

In some aspect the first adapter part is provided with at least one protrusion and the second adapter part is provided with at least one groove for creating a dovetail connection when the first and second adapter parts are connected.

In some aspect the locking means comprises at least a first locking part and a second locking part, wherein the first locking part is provided on the first adapter part and the second locking part is provided on the second adapter part.

In some aspect the first locking part is a resilient pressing piece and the second locking part is a hole adapted for collecting the resilient pressing piece when the first and second adapter parts are connected.

In some aspect the system further comprises at least one flexible piece provided between the second part and the prosthetic limb when attaching the second part onto the prosthetic limb.

In one aspect the system further comprises at least one communication unit for communicating with at least one external device.

In some aspect the at least one communication unit is provided on one or several of the cover, the first part and/or the second part.

In some aspect the locking means comprises magnets adapted for locking the first and second adapter part when they are connected.

In some aspect the at least first cover comprises a compartment adapted for carrying an external device.

In some aspects both of the first adapter part and the second adapter part are provided with at least two holes for engaging at least two screws.

In some aspects the at least two screws are provided for fastening the first adapter part (onto the second adapter part.

In some aspects the at least two screws are provided for adjusting the angle between the prosthetic limb and the adapter device.

In some aspect the first locking part is a resilient pressing piece and the second locking part is a resilient pressing piece wherein the first and second adapter parts are connected and provides a double lock.

The object is obtained by an adapter device for fixing a cover to a prosthetic limb of a prosthesis system, comprising:
a first adapter part configured for being attached to the cover;
a second adapter part configured for being mounted onto the prosthetic limb; and
a locking means for locking the first adapter part onto the second adapter part when the first and second adapter parts are connected.

In some aspect the first adapter part is provided with least one protrusion and the second adapter parts is provided with at least one groove for creating a dovetail connection when the first and second adapter parts are connected.

In some aspect the locking means comprises at least a first locking part and a second locking part, wherein the first locking part is provided on the first adapter part and the second locking part is provided on the second adapter part.

In some aspect the first locking part is a resilient pressing piece and the second locking part is a hole adapted for collecting the resilient pressing piece when the first and second adapter parts are connected.

In some aspect the locking means comprises magnets adapted for locking the first and second adapter part when they are connected.

In some aspect a connecting surface of the first adapter part is inclined and a corresponding connecting surface of the second adapter part is inclined in the opposite direction according to the first adapter part.

In some aspect one or both of the first adapter part and second adapter part comprises a compartment adapted for carrying a communication unit.

In some aspects both of the first adapter part and the second adapter part are provided with at least two holes for engaging at least two screws.

In some aspects the at least two screws are provided for fastening the first adapter part onto the second adapter part.

In some aspects the at least two screws are provided for adjusting the angle between the prosthetic limb and the adapter device.

In some aspects the first locking part is a resilient pressing piece and the second locking part is a resilient pressing piece wherein the first and second adapter parts are connected and provides a double lock.

An advantage of the present disclosure is to offer persons with prostheses increased confidence and improved quality of life. A further advantage is to provide a safe and easy way of connecting and changing a cover to a prosthetic limb. The present disclosure further provides a solution for connecting the prosthetic system to externa communication devices. The price and lead time for custom-made cosmetic solutions for covers to prosthetic limbs are reduced dramatically and could ultimately lead to a commercially viable model.

### BRIEF DESCRIPTION OF THE DRAWINGS

The foregoing will be apparent from the following more particular description of the example embodiments, as illustrated in the accompanying drawings in which like reference characters refer to the same parts throughout the different views. The drawings are not necessarily to scale, emphasis instead being placed upon illustrating the example embodiments.

FIG. 1 illustrates an example of a system according to the present disclosure;

FIG. 2a illustrates an example of the adapter device according to the present disclosure;

FIG. 2b illustrates an example of the adapter device according to the present disclosure;

FIG. 3 illustrates an example of a first adapter part of the adapter device according to the present disclosure;

FIG. 4a illustrates an example of a second adapter part of the adapter device according to the present disclosure;

FIG. 4b illustrates an example of a second adapter part of the adapter device according to the present disclosure;

FIG. 5a illustrates a cut-through view of the adapter device according to the present disclosure;

FIG. 5b illustrates a cut-through view of the adapter device according to the present disclosure;

FIG. 5c illustrates an example of a second adapter part of the adapter device according to the present disclosure;

FIG. 5d illustrates an example of the adapter device according to the present disclosure;

FIG. 5e-f illustrates a cut-through view of the adapter device according to the present disclosure;

FIG. 5g illustrates an example of a second adapter part of the adapter device according to the present disclosure;

FIG. 5h, illustrates an example of the adapter device according to the present disclosure;

FIG. 6 illustrates an example of a system according to the present disclosure;

FIG. 7 is a flow chart illustrating embodiments of method steps according to the present disclosure;

FIG. 8 an example of the adapter device according to the present disclosure;

FIG. 9 an example of the adapter device according to the present disclosure.

FIG. 10 and example of the second adapter part according to the present disclosure.

### DETAILED DESCRIPTION

Aspects of the present disclosure will be described more fully hereinafter with reference to the accompanying drawings. The system, method and device disclosed herein can, however, be realized in many different forms and should not be construed as being limited to the aspects set forth herein. Like numbers in the drawings refer to like elements throughout.

The terminology used herein is for the purpose of describing particular aspects of the disclosure only, and is not intended to limit the disclosure. As used herein, the singular forms “a”, “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

Some of the example embodiments presented herein are directed towards prosthetic covers. The disclosure relates to a prosthesis system adapted for being worn by a user comprising at least a first cover, a prosthetic limb and an adapter device for fixing the first cover to the prosthetic limb wherein the adapter device comprises a first adapter part configured for being attached to the first cover, a second adapter part configured for being mounted onto the prosthetic limb and locking means for locking the cover onto the prosthetic limb. As part of the development of the example embodiments presented herein, a problem will first be identified and discussed.

The prostheses available on the market today are mainly focused on function rather than aesthetics, aiming to provide physical quality of life for the user while the psychological aspects connected to use of prostheses are often given less attention. The possibilities of changing the prosthetic appearance as a way of expressing personality and style or depending on activity and situation are few.

The disclosed prosthetic system provides individually designed cosmetic covers to existing prosthetic limbs. The covers may be manufactured by using a 3D print process, e.g. Selective Laser Sintering, SLS. A solution including prosthetic covers with an adapter device for fixing the cover to the prosthetic limb that provides a quick and easy way of changing prosthetic covers when the user wishes. For example, the user may have a pair of covers that are striped with reflective film and are extremely light to be used when out running, and a more graceful colour matched cover to a dress to switch to when going to a party.

The uniqueness of the disclosed prosthetic system is to enable personalized prostheses at low cost by developing a software that automates parts of the design process, taking into account the technical requirements and helps the user to quickly and easily find a personal expression. This ability to adapt is unique not only in prosthetics, but in all consumer products. The only form of customization that is available today is that the customer can choose between prefabricated components in a modular system, or they can choose colours and materials.

Some users may perform different physical activities, which means that the covers easily get bumps and they should not easily and unwillingly fall off. Thus it is an objective of the invention to provide a safe and stable way of fastening the cover to the prosthetic limb.

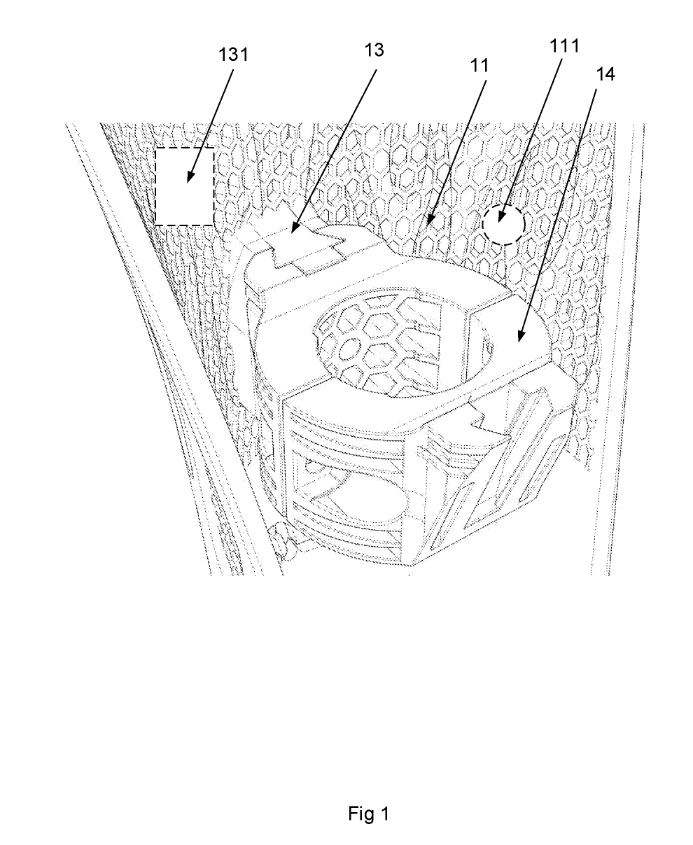

FIG. 1 illustrates a prosthetic system 10 including an adapter device 13, 14 for fixing a cover 11 to a prosthetic limb 12. The adapter device comprises a first adapter part 13 configured for being attached to the cover, a second adapter part configured for being mounted onto the prosthetic limb and a locking means for locking the first adapter part onto the second adapter part when the first and second adapter parts are connected when fixing the cover to the prosthetic limb.

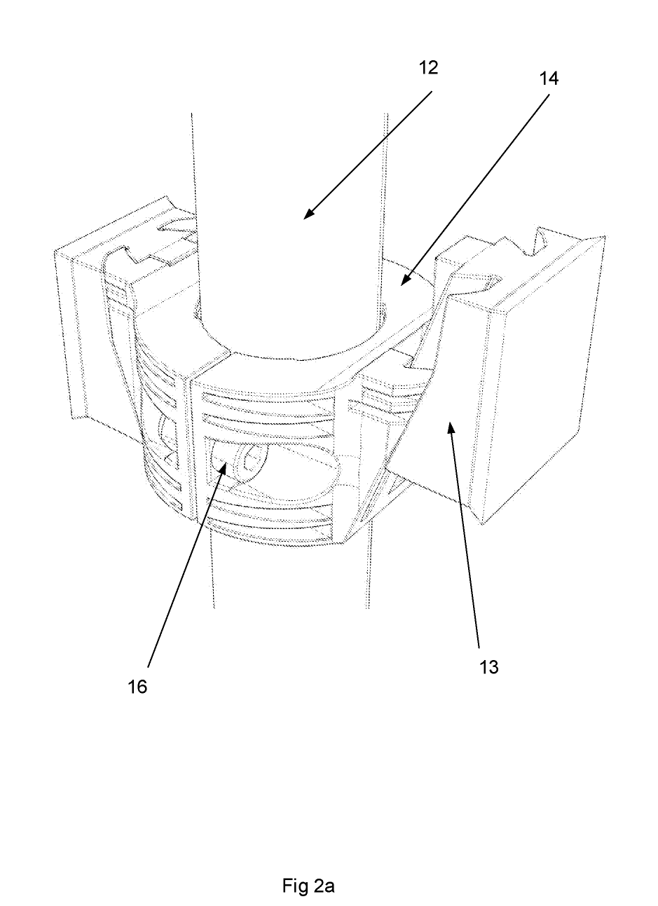

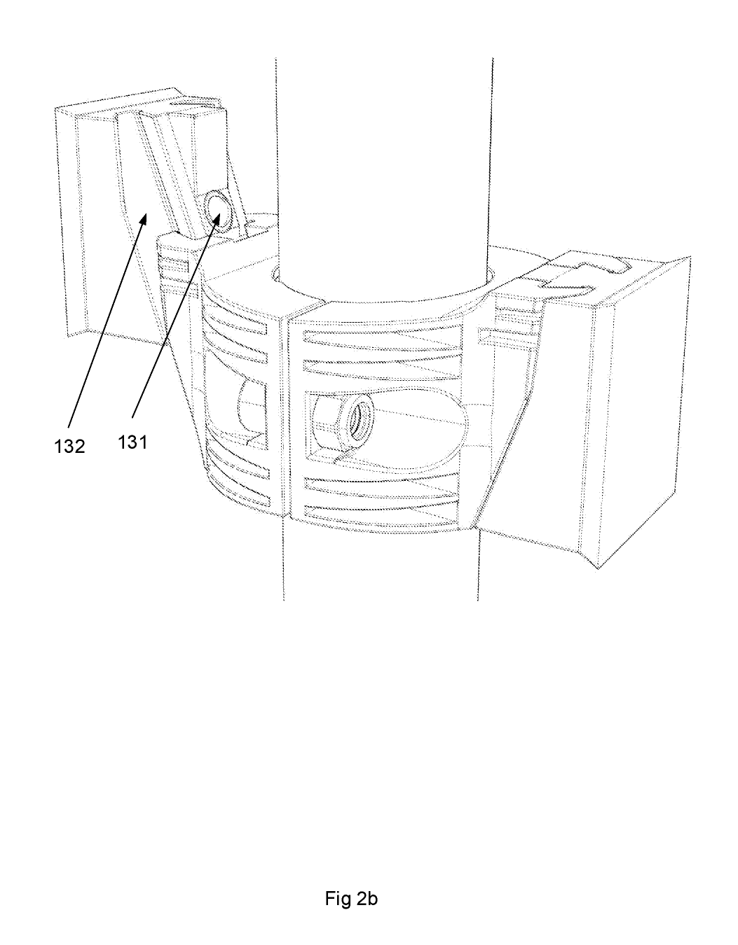

FIG. 2a, illustrates an example of the basic geometry of the system after mounting with at least two fastening means 16 including e.g. a screws and low stop nut, against a tube of the prosthetic limb 12 of e.g. Aluminium. The same fastening means 16 is used on both sides.

FIG. 2a-b, 3, illustrates an example of a first adapter part 13 of the adapter device. This part may be injection moulded to keep the unit cost down and to obtain better strength. However, the part 13 may also be made by the same 3D print process as used for manufacturing the covers. In some aspect, the first adapter part 13 is manufactured together with the cover 11 as one unit. In some aspect, the first adapter part 13 is manufactured as a separate adapter part configured to be attached to the cover 11 before fixing the cover to the prosthetic limb.

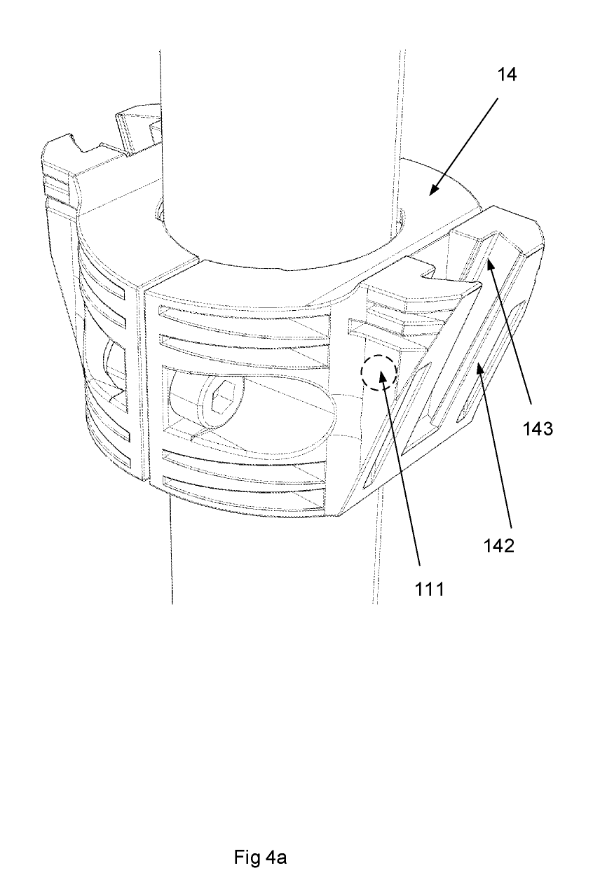

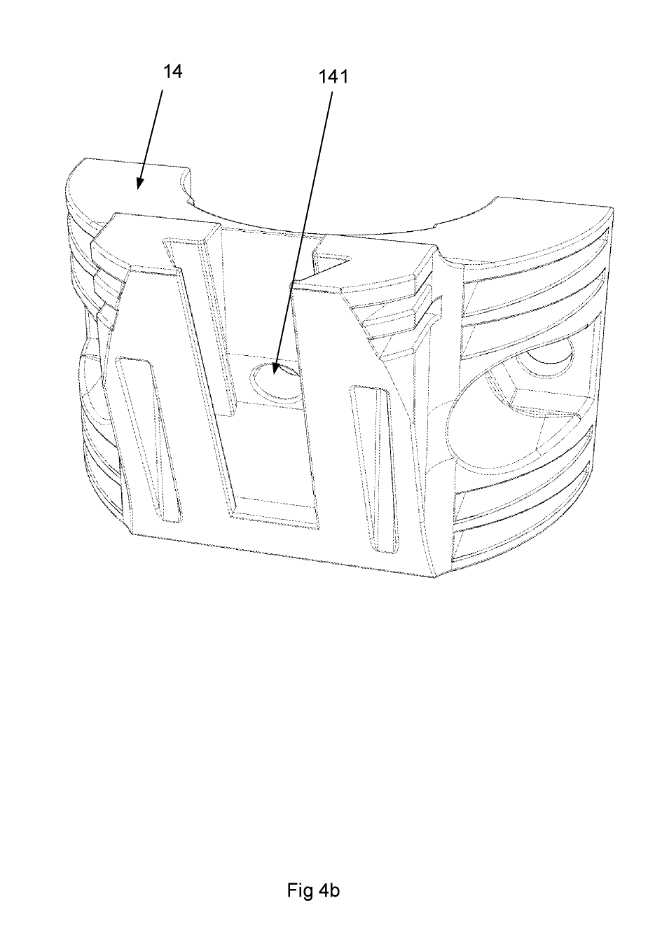

According to some aspect, a connecting surface 132, as shown in FIG. 2b, of the first adapter part 13 is inclined and a corresponding connecting surface 142, as shown in FIG. 4a, of the second adapter part 14 is inclined in the opposite direction according to the first adapter part 13. The inclines connecting surfaces provides an easy way of attaching or fixing the cover 11 to the prosthetic limb.

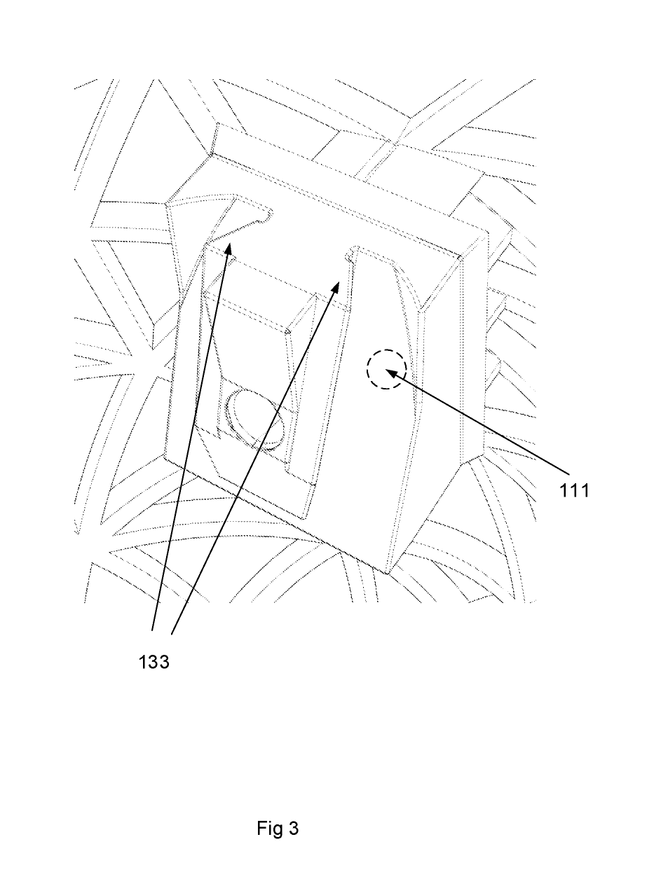

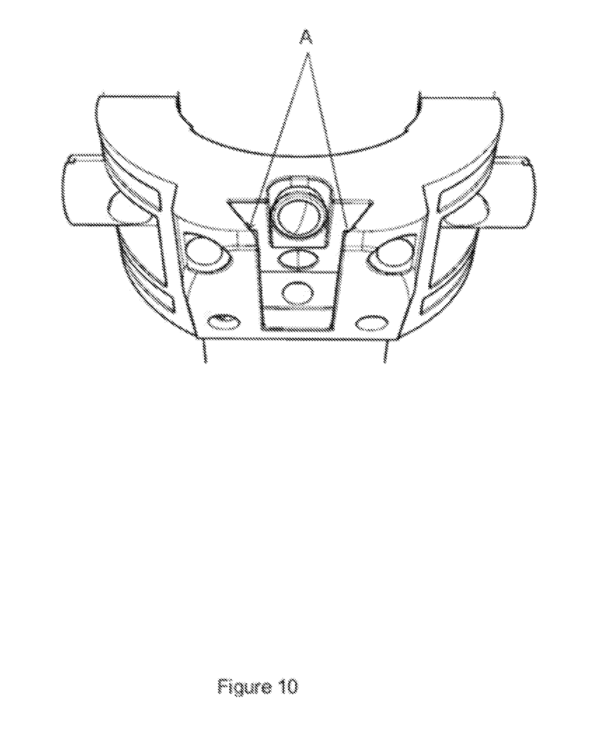

According to some aspects, the first adapter part 13 is provided with at least one protrusion 133, as shown in FIG. 3, and the second adapter parts is provided with at least one groove 143, as shown in FIG. 4a, for creating a dovetail connection when the first and second adapter parts are connected, as shown in FIG. 2a-b. The dovetail connection strengthens the connection between the first adapter part and the second adapter part. In one example, the protrusions are provided along at least two of the edges of the first adapter part 13 and the corresponding grooves are provided along at least two of the edges of the second adapter part 14. In one example, the groves edges are inclined A, as shown in FIG. 10, to minimize the damages on clothes and user when not wearing the cover 11.

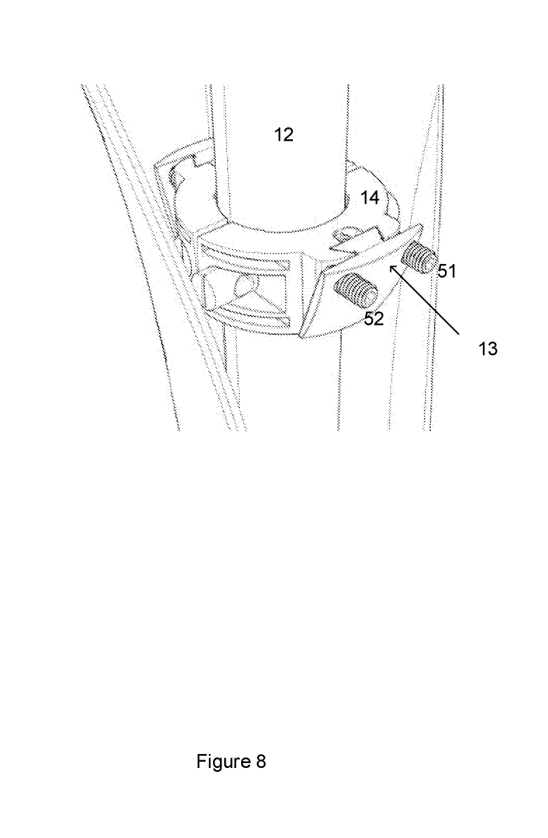

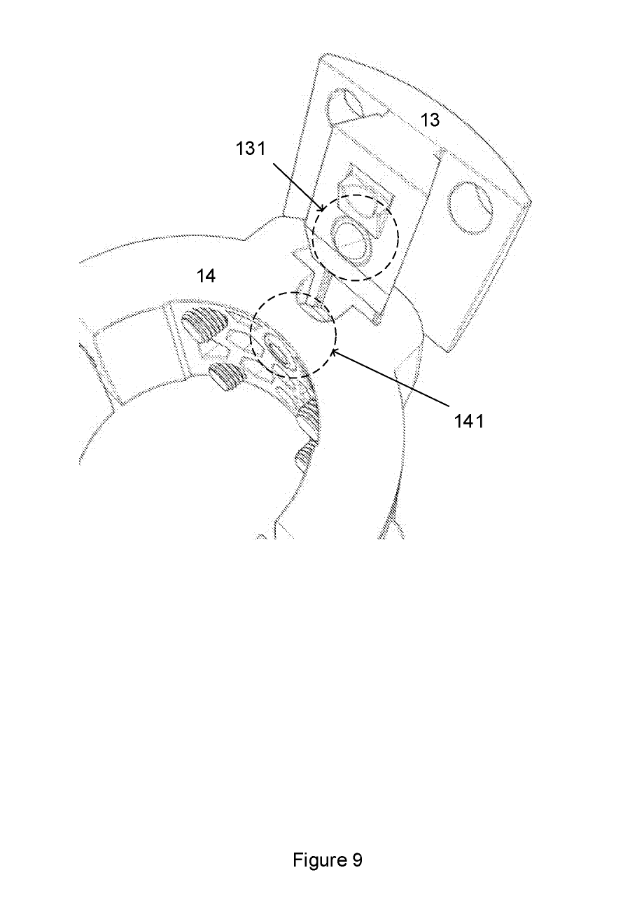

According to some aspects, the locking means comprises at least a first locking part 131, as shown in FIG. 2b, and a second locking part 141, as shown in FIG. 4b. The first locking part 131 is provided on the first adapter part 13 and the second locking part is provided on the second adapter part 14. In some aspect, the first locking part is a resilient pressing piece, e.g. a ball, and the second locking part is a hole adapted for collecting the resilient pressing piece when the first and second adapter parts are connected. In some aspects, the locking means comprises magnets adapted for locking the first and second adapter part when they are connected. In some aspect, as shown in FIG. 8, the first locking part 131 is a resilient pressing piece and the second locking part 141 is a resilient pressing piece and thus there is provided a double lock.

The principle is to provide an intuitive “click” such that the user knows when the cover is in the right position. Since the ball is connected to a spring, not shown, it is then resilient mounted.

The ball will then always be under pressure and thus avoid chatter and noise that may otherwise be caused by vibrations when walking. The force of the spring may be pre-set to be able to determine how easy or hard the cover is fixed.

FIGS. 2a and 2b illustrates the principle of dovetail, where the first adapter part 13 slides down to the bottom of a grove or track provided in the second adapter part 14. FIG. 2b illustrates where a resilient pressing piece, e.g. a ball, slides into a cavity 141, e.g. a hole, provided in the second adapter part 14, and locks the first adapter part 13 onto the second adapter part 14 and thus locks the parts together.

According to some aspects, as shown in FIGS. 3 and 4a, one or both of the first and second adapter parts 13, 14 comprises a compartment adapted for enclosing or carrying a communication unit 111.

The first adapter part 13 may also act as a holder for e.g. a GoPro camera or other functional device etc.

FIGS. 1 and 6 illustrates a prosthesis system 10 adapted for being worn by a user 1 comprising at least one cover 11 and a prosthetic limb 12. The system 10 further comprises the adapter device 13, 14 for fixing the cover 11 to the prosthetic limb 12. The adapter device comprises a first part 13 configured for being attached to the cover, a second part 14 configured for being mounted onto the prosthetic limb 12 and locking means 131, 141 for locking the first adapter part 13 onto the second adapter part 14 when the first and second adapter parts are connected.

The prosthetic limb 12 comprises a tube 121 and a prosthetic foot or hand.

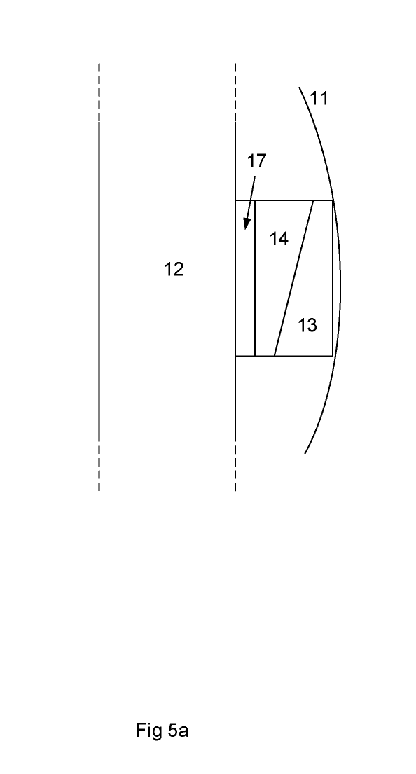

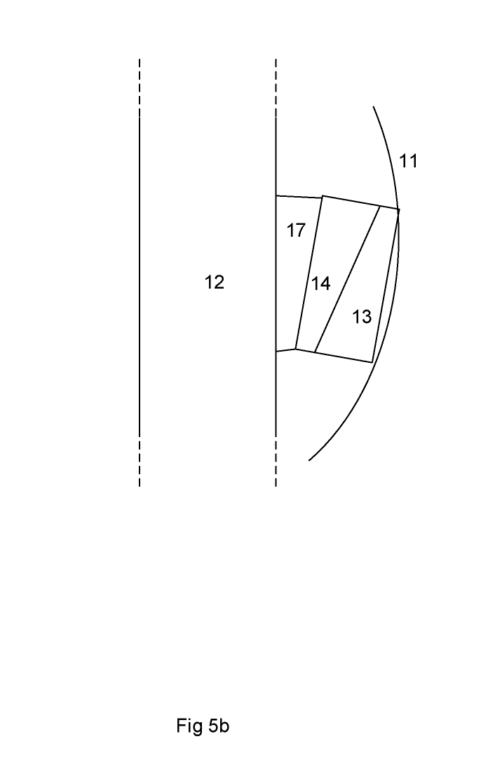

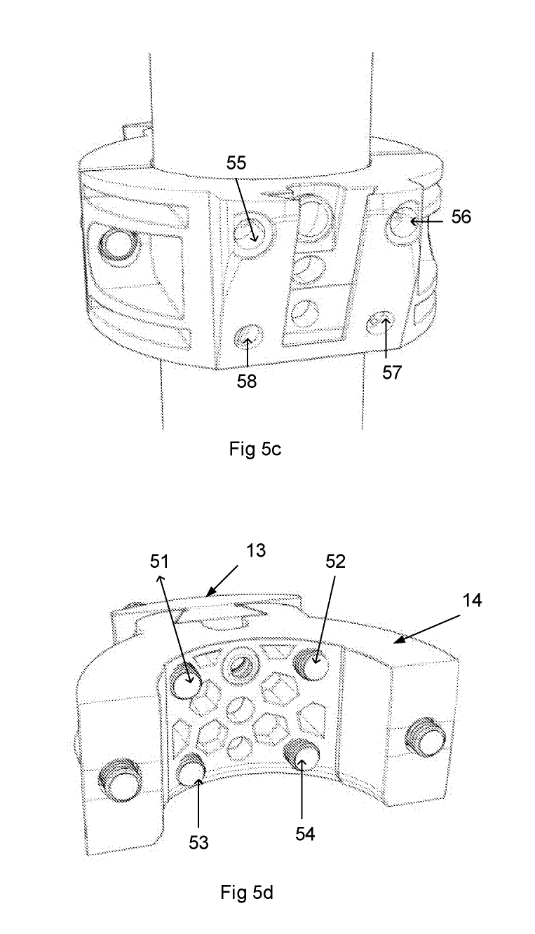

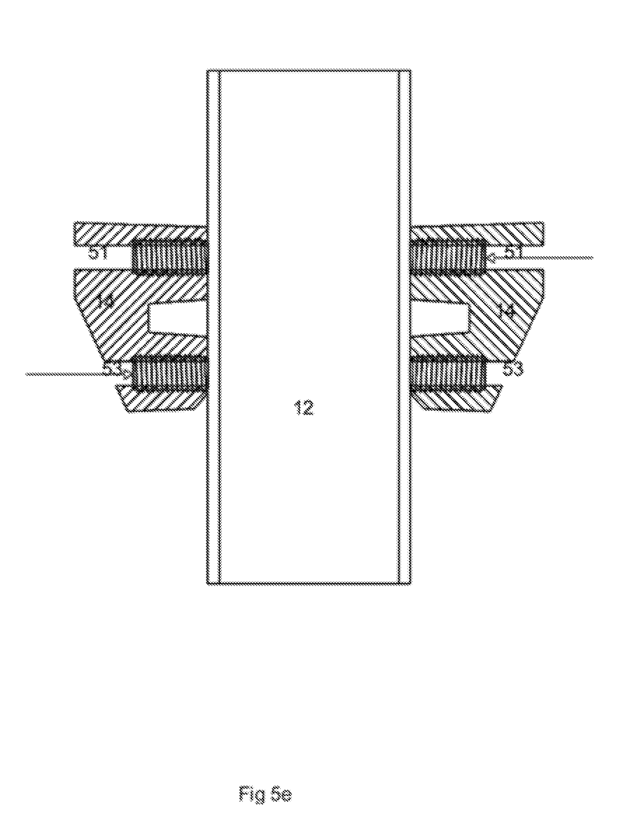

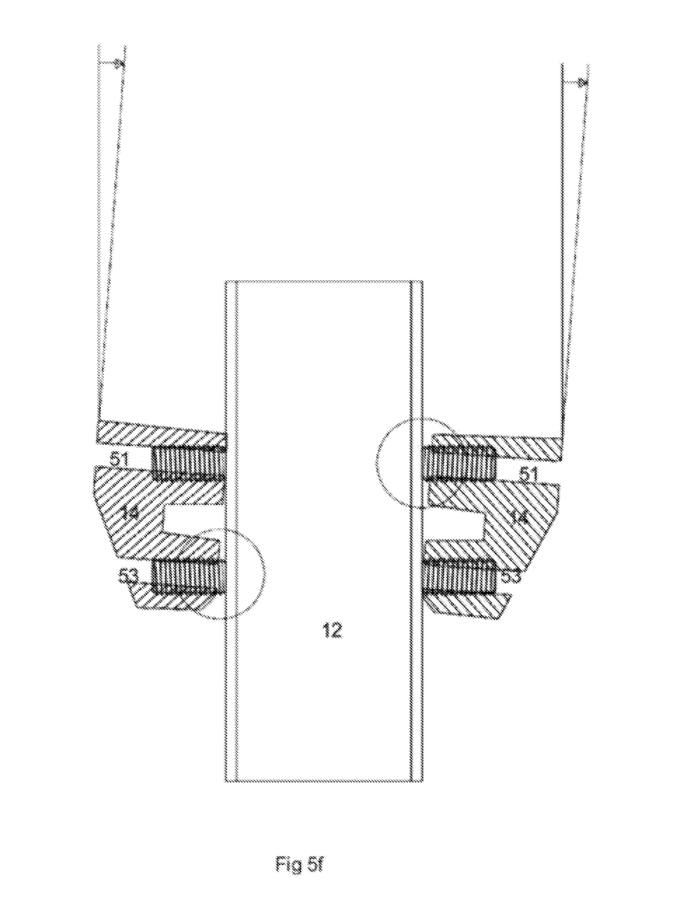

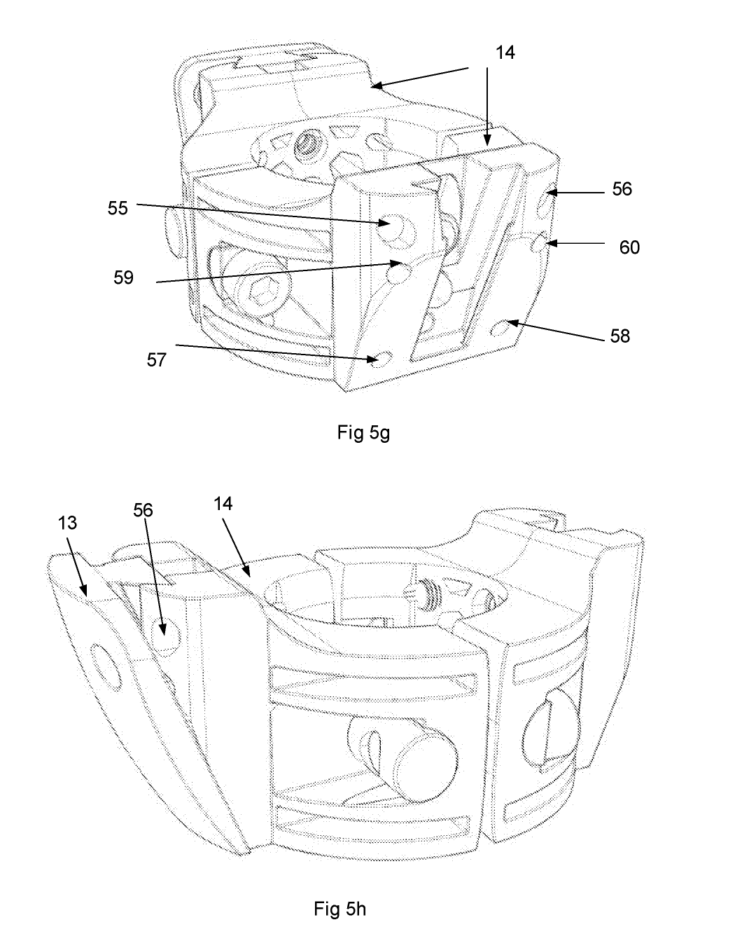

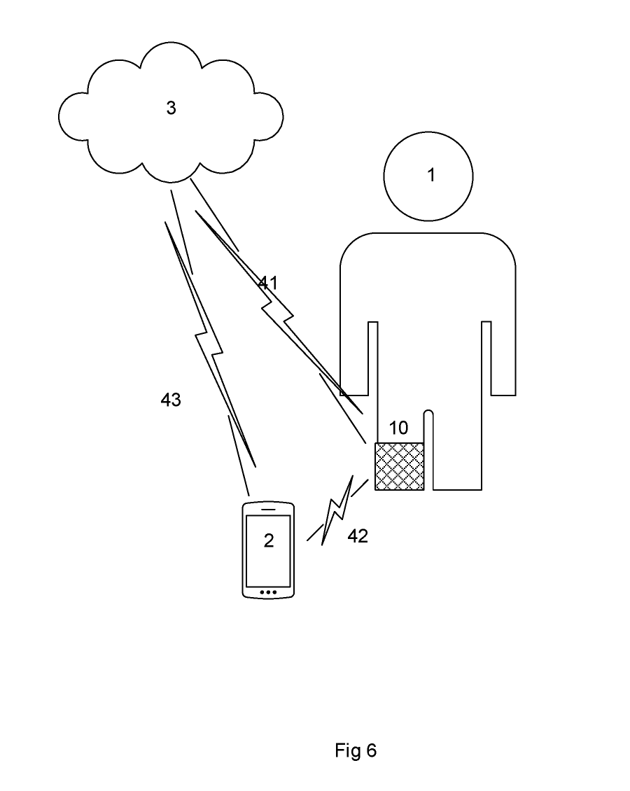

FIGS. 5a and 5b, shows derailment of material for applying a flexible piece 17 e.g. a rubber sheet between the adapter device 13, 14 and the tube of the prosthetic limb 12. The mission of the flexible piece 17 is to contribute to increased friction to prevent slippage, but also to adapt to different dimensions of the tube 121. The diameter of the tube between 30-34 mm is most common. The flexible piece may also be wedge-shaped, as shown in FIG. 5b, to be used to change the angle of the cover 11 if the user or prosthetist has changed the settings of the prosthesis, and it is necessary to compensate. The flexible piece also works as a cushion to prevent breakage on impact against the cover.

According to some aspects, the prosthesis system further comprises at least four screws 51, 52, 53, 54, with associated holes 55, 56, 57, 58, 59, 60 as shown in FIGS. 5c to 5h.

According to some aspects, the screws are used to adjust or change the angle of the cover 11 when attached to the first adapter part 13. The adjustment provides an enhanced and flexible solution for the user. The screws provide adjustments of the angle in the sagittal plane. In one aspect four holes are provided, 55, 56, 57, 58, in the second adapter part 14, as shown in 5c, where four associated screws 51, 52, 53, 54 are used for adjusting the angle. In one aspect, two screws are used for adjusting the angle, as shown in FIGS. 5e and 5f, where FIG. 5e shows a side view with an unchanged angle and FIG. 5f shows a side view with adjusted the angle, the angel has been adjusted by adjusting at least two screws.

According to some aspects, at least two holes 55, 56 are provided through both the first adapter part 13 and the second adapter part 14, as shown in FIG. 5h, where the associated screws 51, 52 are used to fastening the first adapter part 13 onto the second adapter part 14, as shown in FIG. 8. The fastening aspect provides a safe and more secure solution for the user, especially for users that are active and would like to be confident with that the prosthesis will stay in place even when they are active.

FIG. 5g-5h shows an example where two holes are provided through both the first adapter part 13 and the second adapter part 14, and the associated screws are used for fastening the fastening the first adapter part 13 onto the second adapter part 14. There are also provided four holes in the second adapter part 14, with associated screws (not shown), which are used for adjustment of the angle.

According to some aspects, the prosthesis system 10 further comprises at least one communication unit 111 for communicating with at least one external device 2, 3, e.g. a mobile phone 2 or a cloud service 3. In some aspect, the communication unit is configured to communicate directly 42 with at least one external device 2, such a mobile phone, or via 42, 43 a cloud service 3 or directly 41 with the cloud service 3. According to some aspects, at least one communication unit 111 is provided on one or several of the cover 11, the first adapter part 13 and/or the second adapter part 14. One example of a communication unit is pedometers that are linked to an app that gives indication to an orthopaedic engineer or user, or both, that it is time for service and maintenance of the prosthesis. Further examples of functions connected to the communication unit are wireless payment, access e.g. open locks, hotel key, bus card, distress calls e.g. emergency calls, GPS, accelerometer.

According to some aspects, the at least one cover 11 comprises a compartment 113 adapted for carrying an external device, e.g. keys, credit cards, money etc. In one example the compartment is a pocket for carrying wireless communication device, e.g. a smartphone, music player, keys etc.

A “wireless communication device” as the term may be used herein, is to be broadly interpreted to include a radiotelephone having ability for Internet/intranet access, web browser, organizer, calendar, a camera (e.g., video and/or still image camera), a sound recorder (e.g., a microphone), and/or global positioning system (GPS) receiver; a personal communications system (PCS) user equipment that may combine a cellular radiotelephone with data processing; a personal digital assistant (PDA) that can include a radiotelephone or wireless communication system; a laptop; a camera (e.g., video and/or still image camera) having communication ability; and any other computation or communication device capable of transceiving, such as a personal computer, a home entertainment system, a television, etc. Furthermore, a device may be interpreted as any number of antennas or antenna elements.

A software-based design tools is provided to automate and simplify the process from 3D scanning to 3D printing, thus bringing down the cost of cosmetic solutions for prostheses. The software must consider the strength of the prosthetic cover and the material cost, the existing prosthesis and interaction with it and the surrounding tissue and enable the user or designer to control the shape.

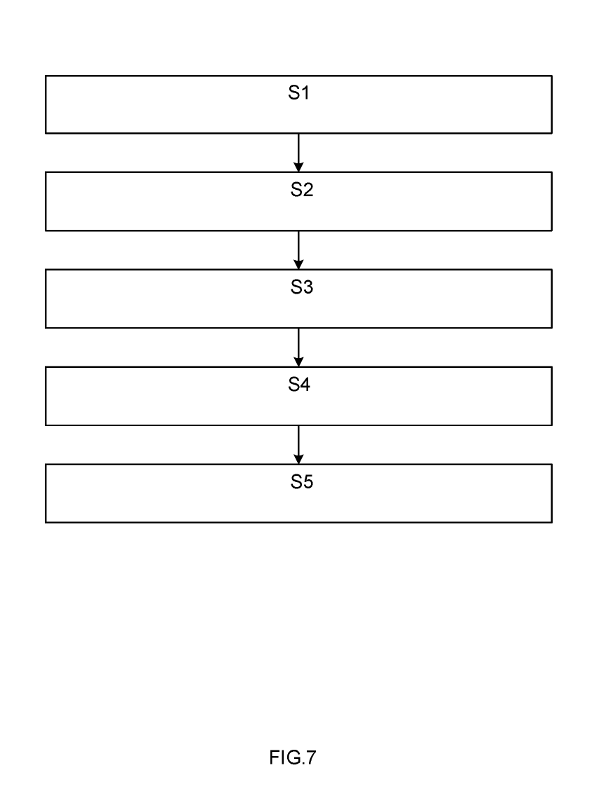

FIG. 7 illustrates the steps of process for manufacturing the described prosthesis system.

Firstly S1, performing a 3D scan of the geometry of the healthy limb and prosthetic limb 2 of a user. The profile of the healthy limb is traced by a CAD program. In some aspects, the healthy limb and the prosthetic limb are measured at some predetermined positions. The measurements are then provided to a program that auto generates a 3D geometry of the healthy limb.

Secondly S2, creating a reflection of the scanned geometries.

Thirdly S3, completing the design of at least one cover 11 of the prosthesis system.

As an example, when the user wear tight pants it looks like he/she has two real legs, instead of showing a glimpse of the profile of the aluminium tubes 121 which usually are the backbone of the prosthetic limb.

Fourthly S4, sending the scanned 3D file to a 3D printer for printing of the cover. The 3D printer prints the covers by using e.g. SLS technology. SLS technology is a manufacturing method that provides the best strength of a 3D printed product, such the disclosed prosthesis system.

Fifthly S5, applying a coat of paint and varnish. There are several ways to add the final surface finish, depending on what effect to achieve. Hydro dipping is a method of achieving a more graphic effect of various patterns applied to the surface via a water bath. Another way is to dress up the covers, for example, leather or textiles in order to achieve a more “dressed” effect. Additional techniques to achieve, for example, a more quality finish may be achieved by using NC milling of aluminium or wood.

According to some aspects, the communication between the communication unit 111 and the external communication device 2, 3 is encrypted. The communication between the communication unit 111 and the external communication device 2, 3 is for example encrypted using Secure Sockets Layer, SSL, or Transport Layer Security, TLS. SSL uses a combination of encryption using public keys and symmetrical keys. SSL may use several different methods of encryption, such as Data Encryption Standard, DES, Digital Signature Algorithm, DSA, Key Exchange Algorithm, KEA, Message-Digest algorithm 5, MD5, Rivest Cipher 2, RC2, Rivest Cipher4, RC4, Rivest, Shamir and Adleman encryption, RSA, Secure Hash Algorithm, SHA-1, SKIPJACK or Triple Data Encryption Standard, Triple-DES.

It should be appreciated that the operations shown in FIG. 7 need not be performed in order. Furthermore, it should be appreciated that not all of the operations need to be performed. Also, the functions or steps noted in the blocks can according to some aspects of the disclosure be executed continuously in a loop.

The various example embodiments described herein are described in the general context of method steps or processes, which may be implemented in one aspect by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Generally, program modules may include routines, programs, objects, components, data structures, etc. that performs particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

The description of the aspects of the disclosure provided herein has been presented for purposes of illustration. The description is not intended to be exhaustive or to limit aspects of the disclosure to the precise form disclosed, and modifications and variations are possible in light of the above teachings or may be acquired from practice of various alternatives to the provided aspects of the disclosure. The examples discussed herein were chosen and described in order to explain the principles and the nature of various aspects of the disclosure and its practical application to enable one skilled in the art to utilize the aspects of the disclosure in various manners and with various modifications as are suited to the particular use contemplated. The features of the aspects of the disclosure described herein may be combined in all possible combinations of methods, apparatus, modules, systems, and computer program products. It should be appreciated that the aspects of the disclosure presented herein may be practiced in any combination with each other.

It should be noted that the word “comprising” does not necessarily exclude the presence of other elements or steps than those listed. It should further be noted that any reference signs do not limit the scope of the claims.

In the drawings and specification, there have been disclosed exemplary aspects. However, many variations and modifications can be made to these aspects. Accordingly, although specific terms are employed, they are used in a generic and descriptive sense only and not for purposes of limitation, the scope of the embodiments being defined by the following claims.

1. A prosthesis system (10) adapted for being worn by a user (1) comprising at least a first cover (11) and a prosthetic limb (12), wherein the system (10) further comprises:
an adapter device (13, 14) for fixing the first cover (11) to the prosthetic limb (12) comprising:
a first adapter part (13) configured for being attached to the first cover;
a second adapter part (14) configured for being mounted onto the prosthetic limb (12); and
locking means (131, 141) for locking the first adapter part (13) onto the second adapter part (14) when the first and second adapter parts are connected.

2. The prosthesis system (10) according to claim 1, wherein a connecting surface (132) of the first adapter part (13) is inclined and a corresponding connecting surface (142) of the second adapter part (14) is inclined in the opposite direction according to the first adapter part (13).

3. The prosthesis system (10) according to claim 1, wherein the first adapter part (13) is provided with at least one protrusion and the second adapter part (14) is provided with at least one groove for creating a dovetail connection when the first and second adapter parts are connected.

4. The prosthesis system (10) according to claim 1, wherein the locking means comprises at least a first locking part (131) and a second locking part (141), wherein the first locking part (131) is provided on the first adapter part (13) and the second locking part (141) is provided on the second adapter part (14).

5. The prosthesis system (10) according to claim 4, wherein the first locking part (131) is a resilient pressing piece and the second locking part (141) is a hole adapted for collecting the resilient pressing piece when the first and second adapter parts (13, 14) are connected.

6. The prosthesis system (10) according to claim 1, wherein the system further comprises at least one flexible piece (17) provided between the second adapter part (14) and the prosthetic limb (12) when attaching the second adapter part onto the prosthetic limb.

7. The prosthesis system (10) according to claim 1, wherein the system further comprises at least one communication unit (111) for communicating with at least one external device (2, 3).

8. (canceled)

9. The prosthesis system (10) according to claim 1, wherein the locking means (131, 141) comprises magnets adapted for locking the first and second adapter part when they are connected.

10. The prosthesis system (10) according to claim 1, wherein the at least first cover (11) comprises a compartment (113) adapted for carrying an external device.

11. The prosthesis system according to claim 1, wherein both of the first adapter part (13) and the second adapter part (14) are provided with at least two holes (55, 56, 57, 58, 59, 60) for engaging at least two screws (51, 52, 53, 54), wherein the at least two screws are provided for fastening the first adapter part (13) onto the second adapter part (14) for adjusting the angle between the prosthetic limb and the adapter device.

12. (canceled)

13. (canceled)

14. The prosthesis system according to claim 4, wherein the first locking part (131) is a resilient pressing piece and the second locking part (141) is a resilient pressing piece wherein the first and second adapter parts are connected and provides a double lock.

15. An adapter device (13, 14) for fixing a cover (11) to a prosthetic limb (12) of a prosthesis system (10), comprising:
a first adapter part (13) configured for being attached to the cover (11);
a second adapter part (14) configured for being mounted onto the prosthetic limb (12); and
a locking means (131, 141) for locking the first adapter part (13) onto the second adapter part (14) when the first and second adapter parts are connected.

16. The adapter device (13, 14) according to claim 15, wherein the first adapter part is provided with least one protrusion and the second adapter parts is provided with at least one groove for creating a dovetail connection when the first and second adapter parts are connected.

17. The adapter device (13, 14) according to claim 15, wherein the locking means comprises at least a first locking part (131) and a second locking part (141), wherein the first locking part is provided on the first adapter part (13) and the second locking part is provided on the second adapter part (14).

18. The adapter device (13, 14) according to claim 17, wherein the first locking part (131) is a resilient pressing piece and the second locking part (141) is a hole adapted for collecting the resilient pressing piece when the first and second adapter parts are connected.

19. The adapter device (13, 14) according to claim 15, wherein the locking means comprises magnets adapted for locking the first and second adapter part when they are connected.

20. The adapter device (13, 14) according to claim 15, wherein a connecting surface (132) of the first adapter part (13) is inclined and a corresponding connecting surface (142) of the second adapter part (14) is inclined in the opposite direction according to the first adapter part (13).

21. The adapter device (13, 14) according to claim 15, wherein one or both of the first adapter part (13) and second adapter part (14) comprises a compartment adapted for enclosing a communication unit (111).

22. The adapter device (13, 14) according to claim 15, wherein both of the first adapter part (13) and the second adapter part (14) are provided with at least two holes (55, 56, 57, 58, 59, 60) for engaging at least two screws (51, 52, 53, 54), wherein the at least two screws are provided for fastening the first adapter part (13) onto the second adapter part (14) for adjusting the angle between the prosthetic limb and the adapter device.

23. (canceled)

24. (canceled)

25. The adapter device (13, 14) according to claim 17, wherein the first locking part (131) is a resilient pressing piece and the second locking part (141) is a resilient pressing piece wherein the first and second adapter parts are connected and provides a double lock.
